(19) United States
(12) Plant Patent Application Publication
(10) Pub. No.: US 2004/0098774 P1
(43) Pub. Date: May 20, 2004

(54) Globularia plant named 'His Eyeball Comp'

(75) Inventor: David Kagan, Moshav Sitrya (IL)

Correspondence Address: Russell D.Orkin, 700 Koppers Building, 436 Seventh Avenue, Pittsburgh, PA, 15219-1818, US

(73) Assignee: Hishtil Afula Ltd.

(21) Appl. No.: 10/298,721

(22) Filed: Nov. 18, 2002

(51) Int. Cl. A01H 5/00

(52) U.S. Cl. PLT/226

(57) ABSTRACT

A new and distinct Globularia arabica Jaub. et Sp. plant with a compact growth habit and dark blue flowers suitable for use as a potted plant.

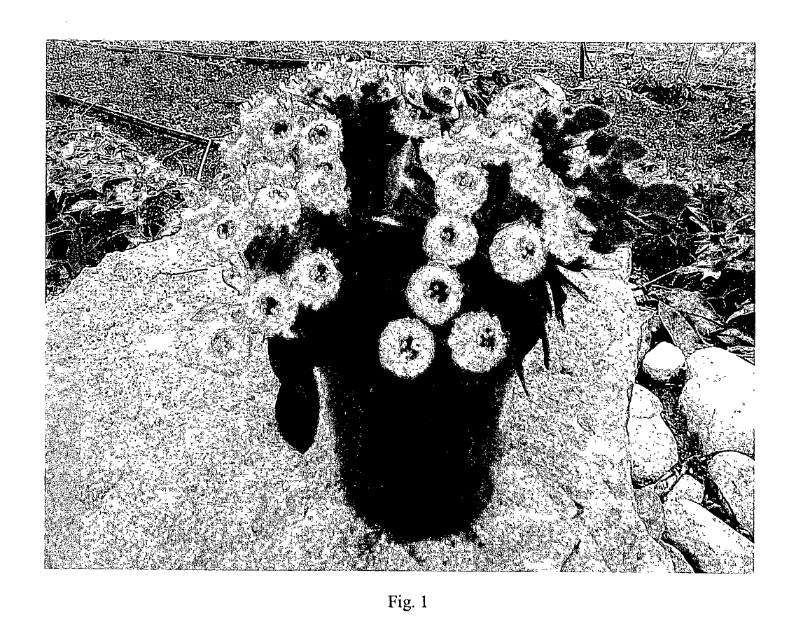

### BOTANICAL CLASSIFICATION

Globularia arabica Jaub. et Sp.

### VARIETAL DENOMINATION

‘His Eyeball Comp’

### BACKGROUND OF THE INVENTION

The present invention comprises a new and distinct cultivar of Globularia arabica Jaub. et Sp. known by the varietal name ‘His Eyeball Comp’. The new variety was discovered in a planned breeding program conducted by the inventor in Moshav Sattarya, located in the central Coastal Plain, Israel.

‘His Eyeball Comp’ originated from a cross made by the inventor of a globularia selection identified as ‘B-5’, not patented, as female parent with a globularia selection identified as ‘21/5’, not patented, as male parent. The new globularia was selected by the inventor, from the seedling population obtained from this cross in 1996 on the basis of its particular compact growth habit, making it suitable as a pot plant.

Asexual propagation of the new plant by terminal cuttings since 1997 has shown that the unique features of the new globularia are stable and propagated true to type in successive generations. The phenotype may vary somewhat due to variation in environment, such a temperature, light intensity, daylength, or fertility level of soil. As globularia can be rooted easily in various environments, no specific conditions for its propagation are required.

### DESCRIPTION OF THE DRAWINGS

The accompanying photographic drawing illustrates the new variety, with the color being as nearly true as is possible with color illustrations of this type.

### DESCRIPTION OF THE PLANT

The following detailed description sets forth the characteristics of the new variety. The data which defines these characteristics were collected by asexual reproductions by cuttings carried out in Sattarya, Israel. The new variety is an ornamental perennial shrub and grows well in sandy soils, open fields, or a greenhouse. Color references are primarily to the R.H.S. Colour Chart of The Royal Horticultural Society of London.

### PLANT

Growth habit: Bushy.

Height: Approximately 20 cm.

Width: Approximately 30 cm.

Growth: Vigorous; slow growth rate.

Branching: Basal.

Flowering habit: Continuous.

Natural flowering season: Spring-summer.

Stems:

Appearance.—Weedy.

Length.—15-30 cm.

Node length.—3-15 mm.

Thickness.—Approximately 3 mm.

Texture.—Slightly tribbed.

Shape.—Round.

Color.—144D.

Pubescence.—Glabrous.

Prickles.—None.

Leaves:

Arrangement.—Alternate.

Composition.—Simple.

Length.—60-70 mm.

Width.—15-20 mm.

Texture.—Smooth.

Surface of upper side.—Glabrous.

Surface of lower side.—Glabrous.

Shape.—Overall: Spatulate. Tip: Apiculate. Base: Tapering. Cross section: Straight. Longitudinal axis: Straight.

Margin.—Entire; very slight.

Color.—Upper surface (young): 147B. Lower surface (young): 147B. Upper surface (mature): 147A. Lower surface (mature): 147A. Veins (upper and lower surface): Main vein somewhat lighter.

Petioles.—Length: Sessile.

Stipules.—Not present.

Inflorescence:

Type.—Complex corymb.

Length.—5-6 cm.

Width.—7-8 cm.

Number of flower heads.—3 to 7.

Flower head just before opening of florets:

Shape.—Slightly domed.

Height.—6 mm.

Width.—10 mm.

Color.—103A.

Flower head with several whorls open:

Opening.—From outside.

Duration.—Approximately 10 days.

Fragrance.—None.

Height.—10 mm.

Diameter.—20 mm.

Number of florets.—Numerous.

Number of sepals.—None.

Floret:

Type.—Labiate.

Shape.—Zygomorphic.

Limb.—Color: White. Length: Approximately 5 mm. Width: Approximately 4 mm. Distal part of limb: 3-lobed.

Tube.—Shape of tube: Funnel. Length of tube: Approximately 3 mm. Color of tube: White.

Number of stamen.—Four.

Number of styles.—One.

Bract:

Shape.—Ovate.

Length.—5 mm.

Color.—Light green.

Pubescence.—Very strong.

Peduncle:

Length.—35-40 mm.

Thickness.—1.5 mm.

Color.—147C.

Pubescence.—Very strong.

Stamen:

Length.—6 mm.

Color.—White.

Anther:

Length.—Approximately 1 mm.

Color.—Light yellow.

Color of pollen.—Light yellow.

Style:

Length.—Approximately 1 mm.

Color.—White.

Stigma:

Color.—White.

Seed:

Length.—Approximately 0.7 mm.

Width.—Approximately 0.5 mm.

Color.—Nearly black.

Shape.—Round to elliptic.

Root:

System.—Spreading.

I claim:

1. A new and distinct variety of Globularia arabica Jaub. et Sp. plant substantially as shown and described.
